(19) United States
(12) Patent Application Publication
(10) Pub. No.: US 2024/0158352 A1
(43) Pub. Date: May 16, 2024

(54) NOVEL ADAMANTYL DERIVATIVE OR PHARMACEUTICALLY ACCEPTABLE SALT THEREOF, AND USE THEREOF

(71) Applicant: CAN-Therapy Inc., Seoul (KR)

(72) Inventors: Hyun Seock SHIN, Gyeonggi-do (KR); Kee Dal NAM, Seoul (KR)

(21) Appl. No.: 18/547,822

(22) PCT Filed: Feb. 25, 2022
(86) PCT No.: PCT/KR2022/002764

§ 371 Date: Nov. 27, 2023

(30) Foreign Application Priority Data
Feb. 26, 2021 (KR) 10-2021-0026320
Feb. 22, 2022 (KR) 10-2022-0022750

(51) Int. Cl. C07D 211/60; A61P 35/00; C07C 237/14

(52) U.S. Cl. CPC C07D 211/60; A61P 35/00; C07C 237/14

(57) ABSTRACT

The present disclosure relates to a novel adamantyl derivative or a pharmaceutically acceptable salt thereof, and a use thereof. The adamantyl derivative may be a dipeptidyl peptidase-4 (DPP4) inhibitor and may be utilized in the prevention or treatment of cancer, rheumatoid arthritis, Parkinson's disease, autoimmune diseases, skin diseases, non-alcoholic steatohepatitis, aortic valve contraction, cerebrovascular diseases, or the like. In particular, the adamantyl derivative suppresses DPP4 in a tumor microenvironment (TME) and guides T cells to tumor tissues, thereby killing cancer cells, and thus can be effectively used for preventing or treating cancer or inhibiting cancer metastasis. The cancer may be prostate cancer, thyroid cancer, kidney cancer, carcinoma, endometrial cancer, lung cancer, urinary epithelial cancer, colorectal cancer, breast cancer, rectal cancer, cervical cancer, glioma, colon cancer, head and neck cancer, stomach cancer, liver cancer, pancreatic cancer, testicular cancer, ovarian cancer, blood cancer, skin cancer, brain tumor, etc.

### TECHNICAL FIELD

The present disclosure relates to a novel adamantyl derivative or a pharmaceutically acceptable salt thereof and use thereof.

### BACKGROUND ART

Dipeptidyl peptidase-4 (DPP4) decomposes several important incretins such as glucagon-like peptide-1 (GLP-1) and glucose-dependent insulinotropic polypeptide (GIP), thereby preventing sufficient secretion of insulin. Therefore, it is known that DPP4 inhibitors are serine proteases that promote the dissociation of dipeptides of the proline or alanine group from the N-terminus of proteins and increase insulin secretion by inhibiting the breakdown of GLP-1 and GIP secreted within the intestine.

Clinical trials have shown that DPP4 inhibitors can lower blood sugar levels, increase glucose tolerance, and have no side effects such as weight gain or hypoglycemia. DPP4 inhibitors currently applied clinically include sitagliptin, saxagliptin, vildagliptin, alogliptin, linagliptin, evogliptin, and the like.

Meanwhile, it has recently been reported that DPP4 inhibitors can exhibit anticancer effects by attracting T cells from the tumor microenvironment (TME) to the tumor parenchyma.

T cells are cells that play an important role in the body's immunity and are largely divided into cytotoxic T cells, helper T cells, regulatory T cells, and memory T cells. When an antigen such as a pathogen enters from the outside, helper T cells secrete inflammatory cytokines and the like to activate killer T cells and B cells. The activated killer T cells kill cells infected with pathogens, and B cells secrete antibodies to inhibit the activity of antigens. Recently, attempts have been made to treat cancer by activating the immunoregulatory ability of T cells.

It is known that CXCL10 (chemokine interferon-y inducible protein 10 kDa) is secreted from the TME to exert anti-tumor effects by chemically attracting monocytes, NK cells, and CXCR3+T cells into the tumor parenchyma. Meanwhile, DDP4 is known to modify CXCL10 as a substrate in the TME, and accordingly, research using DDP4 inhibitors to maintain the anti-tumor activity of CXCL10 in the TME is actively underway.

In addition, evogliptin, one of the DPP4 inhibitors, is undergoing clinical trials as a treatment for calcific aortic valve disease, and it has been reported that DPP4 expression is increased in patients with non-alcoholic steatohepatitis (NASH).

Accordingly, the present inventors produced a novel adamantyl derivative, and the present disclosure was completed by confirming that the derivative or a pharmaceutically acceptable salt thereof inhibits DDP4 and induces cell death in cancer cell lines.

### DISCLOSURE OF THE INVENTION

### Technical Goals

The technical aspect to be achieved by the present disclosure is to provide a novel adamantyl derivative, a racemate, isomer, solvate, or pharmaceutically acceptable salt thereof.

Further, another aspect of the present disclosure is to provide a composition for inhibiting DPP4 (dipeptidyl peptidase-4), the composition including the adamantyl derivative, racemate, isomer, solvate, or pharmaceutically acceptable salt thereof as an active ingredient.

Further, still another aspect of the present disclosure is to provide a pharmaceutical composition for preventing or treating one or more diseases selected from the group consisting of cancer, rheumatoid arthritis, Parkinson's disease, autoimmune disease, skin disease, non-alcoholic steatohepatitis, aortic valve constriction, and cerebrovascular disease, the pharmaceutical composition including the adamantyl derivative, racemate, isomer, solvate or pharmaceutically acceptable salt thereof as an active ingredient.

Further, yet another aspect of the present disclosure is to provide a food composition for preventing or alleviating one or more diseases selected from the group consisting of cancer, rheumatoid arthritis, Parkinson's disease, autoimmune disease, skin disease, non-alcoholic steatohepatitis, aortic valve constriction, and cerebrovascular disease, the food composition including the adamantyl derivative or foodologically acceptable salt thereof as an active ingredient.

Further, still another aspect of the present disclosure is to provide a cosmetic composition for preventing or alleviating one or more diseases selected from the group consisting of cancer, rheumatoid arthritis, Parkinson's disease, autoimmune disease, skin disease, non-alcoholic steatohepatitis, aortic valve constriction, and cerebrovascular disease, the cosmetic composition including the adamantyl derivative, or cosmetically acceptable salt thereof as an active ingredient.

Further, still another aspect of the present disclosure is to provide a method of preparing the adamantyl derivative, racemate, isomer, solvate, or pharmaceutically acceptable salt thereof.

However, technical goals to be achieved are not limited to those described above, and other goals not mentioned above are clearly understood by one of ordinary skill in the art from the following description.

### Technical Solutions

In order to address the above issues, the present disclosure provides an adamantyl derivative represented by the following [Chemical Formula 1], a racemate, isomer, solvate, or pharmaceutically acceptable salt thereof.

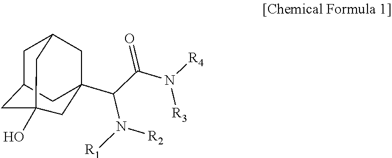

As an embodiment of the present disclosure, in the above [Chemical Formula 1], R₁ and R₂ may be the same or different from each other and be each independently hydrogen, a C₁-C₈ chain or a cyclic alkyl group; R₃ and R₄ may be the same or different from each other and be each independently a C₁-C₈ chain or cyclic alkyl group, a C₁-C₈ alkoxy group, or a hydroxy group, or combine with each other to form a C₃-C₈ single ring heterocycloalkyl group containing N; one or more hydrogens of the C₁-C₈ chain or cyclic alkyl group, C₁-C₈ alkoxy group, or C₃-C₈ single ring heterocycloalkyl group may be each independently unsubstituted or substituted with any one or more substituents selected from the group consisting of a halogen group, a hydroxy group, a cyano group, a nitro group, and a trifluoromethyl group.

As another embodiment of the present disclosure, R₂ and R₃ may be each independently a C₁-C₈ chain alkyl group substituted with a hydroxy group.

As another embodiment of the present disclosure, the single ring heterocycloalkyl group formed by combining R₂ and R₃ may be substituted with the cyano group or the trifluoromethyl group.

As another embodiment of the present disclosure, the adamantyl derivative represented by [Chemical Formula 1] may be any one selected from the group consisting of compounds represented by the following formulas:

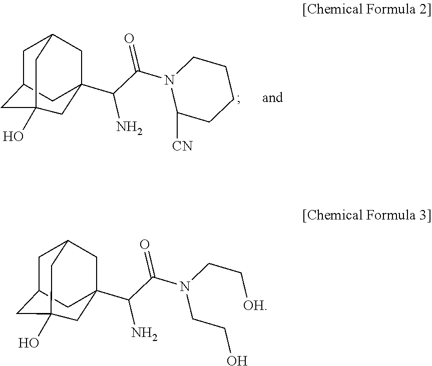 [SMILES: N#CC1CCCCN1C(=O)C(N)C12CC3CC(CC(O)(C3)C1)C2 ; NC(C(=O)N(CCO)CCO)C12CC3CC(CC(O)(C3)C1)C2]

As another embodiment of the present disclosure, the adamantyl derivative, racemate, isomer, solvate, or a pharmaceutically acceptable salt thereof may inhibit DPP4 (dipeptidyl peptidase-4).

Further, the present disclosure provides the composition for inhibiting DPP4 (dipeptidyl peptidase-4), the composition including the adamantyl derivative, racemate, isomer, solvate, or pharmaceutically acceptable salt thereof as an active ingredient.

Further, the present disclosure provides the composition for preventing or treating one or more diseases selected from the group consisting of cancer, rheumatoid arthritis, Parkinson's disease, autoimmune disease, skin disease, non-alcoholic steatohepatitis, aortic valve constriction, and cerebrovascular disease, desirably cancer, the pharmaceutical composition including the adamantyl derivative, racemate, isomer, solvate or pharmaceutically acceptable salt thereof as an active ingredient.

Further, the present disclosure provides the method of preventing or treating one or more diseases selected from the group consisting of cancer, rheumatoid arthritis, Parkinson's disease, autoimmune disease, skin disease, non-alcoholic steatohepatitis, aortic valve constriction, and cerebrovascular disease, desirably cancer, the method including administering the pharmaceutical composition including the adamantyl derivative, racemate, isomer, solvate or pharmaceutically acceptable salt thereof as an active ingredient to a subject.

In addition, the present disclosure provides a use of the adamantyl derivative, racemate, isomer, solvate or pharmaceutically acceptable salt thereof for the production of a drug for preventing or treating one or more diseases selected from the group consisting of cancer, rheumatoid arthritis, Parkinson's disease, autoimmune disease, skin disease, non-alcoholic steatohepatitis, aortic valve constriction, and cerebrovascular disease.

As another embodiment of the present disclosure, the cancer may be any one or more selected from the group consisting of prostate cancer, thyroid cancer, kidney cancer, carcinoma, endometrial cancer, lung cancer, urinary epithelial cancer, colorectal cancer, breast cancer, rectal cancer, cervical cancer, glioma, colon cancer, head and neck cancer, stomach cancer, liver cancer, pancreatic cancer, testicular cancer, ovarian cancer, blood cancer, skin cancer, or brain tumor, but is not limited thereto.

Further, the present disclosure provides the food composition for preventing or alleviating one or more diseases selected from the group consisting of cancer, rheumatoid arthritis, Parkinson's disease, autoimmune disease, skin disease, non-alcoholic steatohepatitis, aortic valve constriction, and cerebrovascular disease, desirably cancer, the food composition including the adamantyl derivative, racemate, isomer, solvate or foodologically acceptable salt thereof as an active ingredient.

Further, the present disclosure provides the cosmetic composition for preventing or alleviating one or more diseases selected from the group consisting of cancer, rheumatoid arthritis, Parkinson's disease, autoimmune disease, skin disease, non-alcoholic steatohepatitis, aortic valve constriction, and cerebrovascular disease, desirably cancer, the cosmetic composition including the adamantyl derivative, racemate, isomer, solvate or cosmetically acceptable salt thereof as an active ingredient.

Further, the present disclosure provides a method of preparing the adamantyl derivative, racemate, isomer, solvate, or pharmaceutically acceptable salt thereof, the method including the following steps.

A method of preparing the adamantyl derivative, or pharmaceutically acceptable salt thereof, including
(1) synthesizing a compound represented by [Chemical Formula 1-3] from the compound represented by [Chemical Formula 2-1] and the compound represented by [Chemical Formula 2-2]; and
(2) dissolving the compound represented by [Chemical Formula 2-3] in acetonitrile, stirring the same with toluenesulfonic acid (TsOH), and filtering the mixture, to obtain an adamantyl derivative represented by [Chemical Formula 2-4].

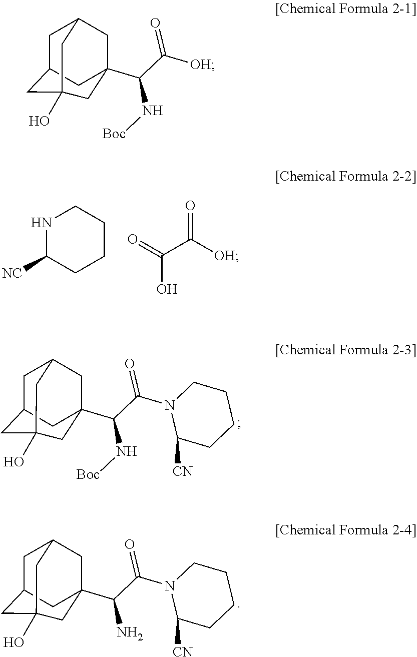

As an embodiment of the present disclosure, the method may include, after step 2, treating the compound represented by [Chemical Formula 2-4] with TFA (trifluoroacetic acid )salt) to obtain a pharmaceutically acceptable salt thereof, and the salt may desirably be represented by [Chemical Formula 2-5].

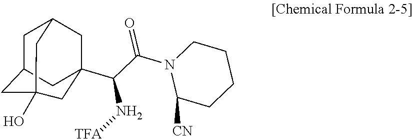

### Effects

The present disclosure relates to a novel adamantyl derivative or a pharmaceutically acceptable salt thereof and its use, the adamantyl derivative may be a DPP4 (dipeptidyl peptidase-4) inhibitor, and may be used to prevent or treat one or more diseases selected from the group consisting of cancer, rheumatoid arthritis, Parkinson's disease, autoimmune disease, skin disease, non-alcoholic steatohepatitis, aortic valve constriction, and cerebrovascular disease. In particular, the adamantyl derivative inhibits DPP4 in the tumor microenvironment (TME) to attract T cells to tumor tissue, thereby killing cancer cells, which may be useful for preventing or treating cancer, or inhibiting metastasis of cancer. The cancer may be selected from the group consisting of prostate cancer, thyroid cancer, kidney cancer, carcinoma, endometrial cancer, lung cancer, urinary epithelial cancer, colorectal cancer, breast cancer, rectal cancer, cervical cancer, glioma, colon cancer, head and neck cancer, stomach cancer, liver cancer, pancreatic cancer, testicular cancer, ovarian cancer, blood cancer, skin cancer, brain tumor, and the like.

The effects of the present disclosure are not limited to those mentioned above, and other effects not mentioned will be clearly understood by those skilled in the art from the description below.

### BRIEF DESCRIPTION OF DRAWINGS

FIG. 1 shows the results of confirming DPP4 inhibitory activity after treatment the adamantyl derivatives KUgt102 and KUgt103 of the present disclosure and vildagliptin and saxagliptin, known in the same classes of DPP4 inhibitors, at 1 μM, 100 nM, 10 nM, 1 nM and 100 pM, respectively.

FIG. 2 shows the results of confirming cell viability after treating LLC, a mouse lung carcinoma cell line, with evogliptin and saxagliptin known as DPP4 inhibitors of the same class as the adamantyl derivative (KUgt-102) of the present disclosure.

FIG. 3 shows the results of confirming cell viability after treating A549, a human lung carcinoma cell line, with evogliptin and saxagliptin known as DPP4 inhibitors of the same class as the adamantyl derivative (KUgt-102) of the present disclosure.

### BEST MODE FOR CARRYING OUT THE INVENTION

As a result of intensive research on a novel adamantyl derivative or pharmaceutically acceptable salt thereof, the present inventors confirmed the DPP4 inhibitory activity of the derivatives and completed the present disclosure.

More specifically, it was confirmed that the adamantyl derivative or a pharmaceutically acceptable salt thereof, as a DPP4 inhibitor, has anticancer activity by attracting T cells from the tumor microenvironment to tumor tissue.

From the above results, the present disclosure provides a novel adamantyl derivative. represented by the following [Chemical Formula 1] or pharmaceutically acceptable salt thereof

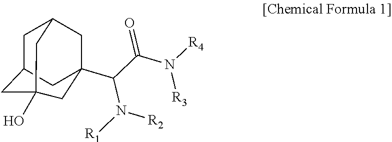

In the above [Chemical Formula 1], R₁ and R₂ may be the same or different from each other and be each independently hydrogen, a C₁-C₈ chain or a cyclic alkyl group; R₃ and R₄ may be the same or different from each other and be independently a C₁-C₈ chain or cyclic alkyl group, a C₁-C₈ alkoxy group, or a hydroxy group, or combine with each other to form a C₃-C₈ single ring heterocycloalkyl group containing N; one or more hydrogens of the C₁-C₈ chain or cyclic alkyl group, C₁-C₈ alkoxy group, or C₃-C₈ single ring heterocycloalkyl group may be each independently unsubstituted or substituted with any one or more substituents selected from the group consisting of a halogen group, a hydroxy group, a cyano group, a nitro group, and a trifluoromethyl group.

In the present disclosure, the “adamantyl derivative” may include an adamantyl derivative represented by the [Chemical Formula 1], or a racemate, isomer, and solvate thereof. Specifically, to the extent that the adamantyl derivative exhibits one or more stereocenters, the adamantyl derivative may be a racemate, an enantiomerically pure or diastereomerically pure compound, or an enantiomerically enriched or diastereomerically enriched compound. The ‘enantiomerically enriched compound’ refers to a compound with an enantiomeric excess (ee) of 50% or more. The ‘diastereomerically enriched compound’ refers to a compound with a diastereomeric excess (de) of 30% or more. However, according to the present disclosure, it may be an optional mixture of diastereomers. In the present disclosure, the single ring heterocycloalkyl group formed by combining R₂ and R₃ may be substituted with a cyano group or trifluoromethyl group, and desirably may be substituted with the (S) configuration.

According to one embodiment of the present disclosure, the adamantyl derivative may be of the following [Chemical Formula 2] or [Chemical Formula 3], desirably [Chemical Formula 2-4] or [Chemical Formula 3-5].

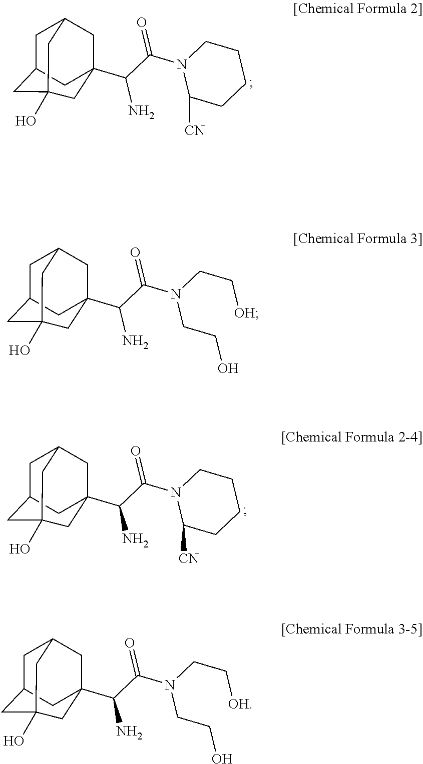 [SMILES: N#CC1CCCCN1C(=O)C(N)C12CC3CC(CC(O)(C3)C1)C2 ; NC(C(=O)N(CCO)CCO)C12CC3CC(CC(O)(C3)C1)C2 ; N#C[C@@H]1CCCCN1C(=O)[C@@H](N)C12CC3CC(CC(O)(C3)C1)C2 ; N[C@H](C(=O)N(CCO)CCO)C12CC3CC(CC(O)(C3)C1)C2]

In the present disclosure, the term “substitution” refers to a reaction that replaces an atom or atom group contained in a molecule of a compound with another atom or atom group.

In the present disclosure, the term “chain” refers to a molecule with a chained structure, and a chained structure is a chemical structure in which carbon atoms are connected in a chain shape and includes straight chain and branched structures. “Cyclic” refers to a ring-shaped structure where both ends of the chain are connected in the skeleton of an organic compound. “Chain or cyclic alkyl group” means a monovalent linear or branched or cyclic saturated hydrocarbon residue consisting only of carbon and hydrogen atoms, having from 1 to 12 carbon atoms.

In the present disclosure, the term “heterocycloalkyl group” usually refers to a saturated or unsaturated (but not aromatic) cyclohydrocarbon, which may be optionally unsubstituted, mono-substituted, or multiply substituted, and at least one in its structure is selected from heteroatoms of N, O or S.

In the present disclosure, the term “alkoxy group” refers to an alkyl group (—O—R) bonded to oxygen. Examples of such alkoxy groups include, but are not limited to, methoxy groups, ethoxy groups, propoxy groups, butoxy groups, etc.

In the present disclosure, the term “pharmaceutically acceptable salt” refers to a formulation of a compound that does not cause serious irritation to the organism to which the compound is administered and does not impair the biological activity and physical properties of the compound. The pharmaceutically acceptable salt may be obtained by reacting the compound of the present disclosure with inorganic acids such as hydrochloric acid, hydrobromic acid, sulfuric acid, nitric acid, and phosphoric acid, sulfonic acids such as methanesulfonic acid, ethanesulfonic acid, and p-toluenesulfonic acid, and an organic carboxylic acid such as tartaric acid, formic acid, citric acid, acetic acid, trichloroacetic acid, trifluoroacetic acid, capric acid, isobutanoic acid, malonic acid, succinic acid, phthalic acid, gluconic acid, benzoic acid, lactic acid, fumaric acid, maleic acid, and salicylic acid. Further, it may be obtained by reacting the compound of the present disclosure with a base to produce alkali metal salts such as ammonium salts, sodium or potassium salts, alkaline earth metal salts such as calcium or magnesium salts, salts of organic bases such as dicyclohexylamine, N-methyl-D-glucamine, and tris(hydroxymethyl) methylamine, and amino acid salts such as arginine and lysine.

In addition, the adamantyl derivative or a pharmaceutically acceptable salt thereof may include not only pharmaceutically acceptable salts, but also all salts, hydrates, and solvates that may be prepared by conventional methods.

The adamantyl derivative of the present disclosure inhibits DPP4 and may be used for the pharmaceutical composition for preventing or treating one or more diseases selected from the group consisting of cancer, rheumatoid arthritis, Parkinson's disease, autoimmune disease, skin disease, non-alcoholic steatohepatitis, aortic valve constriction, and cerebrovascular disease, the pharmaceutical composition including the adamantyl derivative or pharmaceutically acceptable salt thereof as an active ingredient.

In the present disclosure, the term “prevention” refers to all actions that suppress or delay the occurrence, spread, or recurrence of the above-mentioned disease by administration of the composition of the present disclosure, and “treatment” refers to all actions that improve or beneficially change symptoms of the disease by administration of the composition of the present disclosure.

In the present disclosure, the term “pharmaceutical composition” refers to a product prepared for the purpose of preventing or treating a disease and may be formulated and used in various forms according to conventional methods. For example, it may be formulated into oral dosage forms such as powders, granules, tablets, capsules, suspensions, emulsions, syrups, etc., and may be formulated and used in the form of external preparations, suppositories, and sterile injection solutions.

In the present disclosure, “included as an active ingredient” means that the ingredient is included in an amount necessary or sufficient to achieve the desired biological effect. In actual application, the amount included as an active ingredient may be determined by considering the fact that it is the amount to treat the target disease and does not cause other toxicities, for example, it may vary depending on various factors such as the disease or condition being treated, the form of the composition administered, the size of the subject, or the severity of the disease or condition, etc. One or ordinary skill in the art can empirically determine the effective amount of an individual composition without undue experimentation.

In addition, the pharmaceutical composition of the present disclosure may contain one or more pharmaceutically acceptable carriers in addition to the active ingredients described above, depending on each formulation.

The pharmaceutically acceptable carrier may be saline solution, sterile water, Ringer's solution, buffered saline solution, dextrose solution, maltodextrin solution, glycerol, ethanol, and a mixture of one or more of these ingredients, and if necessary, may further include other common additives such as antioxidant, buffer, and bacteriostatic agent. In addition, a diluent, dispersant, surfactant, binder, and lubricant may be additionally added to formulate an injectable formulation such as an aqueous solution, suspension, emulsion, etc., pill, capsule, granule, or tablet. Furthermore, it may be desirably formulated according to each disease or ingredient using an appropriate method in the art or a method disclosed in Remington's Pharmaceutical Science (Mack Publishing Company, Easton PA).

The composition of the present disclosure may be administered orally or parenterally in a pharmaceutically effective amount, depending on the desired method, in the present disclosure, the term “pharmaceutically effective amount” refers to an amount sufficient to treat a disease with a reasonable benefit/risk ratio applicable to medical treatment and not to cause side effects, and the effective dose level may be determined based on well-known factors including the patient's health status, severity, activity of the drug, sensitivity to the drug, method of administration, time of administration, route of administration, excretion rate, duration of treatment, drugs combined or used simultaneously, and other medical fields.

Therefore, the pharmaceutical composition of the present disclosure is administrated to a subject to prevent, treat, and/or diagnose cancer, autoimmune diseases including rheumatoid arthritis, neurodegenerative diseases including Parkinson's disease, skin diseases, non-alcoholic fatty liver disease including non-alcoholic steatohepatitis, metabolic diseases including diabetes, and cerebrovascular diseases including aortic valve constriction, and the diseases may be diseases in which DPP4 is overexpressed in the experimental group compared to the normal group.

There is no limit to the type of the cancer, but non-limiting examples thereof include any one selected from the group consisting of prostate cancer, thyroid cancer, kidney cancer, carcinoma, endometrial cancer, cervical cancer, lung cancer, glioma, colon cancer, rectal cancer, colorectal cancer, head and neck cancer, stomach cancer, liver cancer, pancreatic cancer, urinary epithelial cancer, testicular cancer, breast cancer, ovarian cancer, blood cancer, skin cancer, brain tumor, and the like. The autoimmune disease may be rheumatoid arthritis, Crohn's disease, erythema, Hashimoto's thyroiditis, pernicious anemia, Addison's disease, type 1 diabetes, lupus, chronic fatigue syndrome, fibromyalgia, hypothyroidism and hyperthyroidism, scleroderma, Behcet's disease, and severe myasthenia gravis, etc., but is not limited thereto. The neurodegenerative disease may be Parkinson's disease, Alzheimer's disease, Huntington's disease, multiple sclerosis, allergic myelitis, etc., but is not limited thereto. The skin disease may be atopic dermatitis, psoriasis, erythematous disease triggered by radiation, chemicals, burns, etc., acid burn, acne, etc., but is not limited thereto. The non-alcoholic fatty liver disease may include simple hepatic steatosis, non-alcoholic hepatitis, liver fibrosis, etc. The metabolic diseases may be pre-diabetes, glucose intolerance, pathological fasting glucose, diabetic foot, diabetes-related ulcer, diabetic hyperlipidemia, diabetic dyslipidemia, newly diagnosed type 1 diabetes, gestational diabetes, heart failure, hyperglycemia, postprandial adrenaline syndrome, etc. and may be desirably type 2 diabetes. The cardiovascular disease may desirably be aortic valve constriction.

In the present disclosure, the term “subject” is not limited to any mammal such as livestock or human in need of prevention, treatment, and/or diagnosis of the disease, but is desirably a human.

The pharmaceutical composition of the present disclosure may be formulated in various forms for administration to a subject, and a representative formulation for parenteral administration is an injectable formulation, and an isotonic aqueous solution or suspension is desired. The injectable formulation may be prepared according to techniques known in the art using suitable a dispersing or wetting agent and suspending agent. For example, each component may be dissolved in saline or buffer solution to be formulated for injection. Further, formations for oral administration include, for example, an ingestible tablet, buccal tablet, troche, capsule, elixir, suspension, syrup, wafer, and the like, and these formulations contain a diluent (e.g., lactose, dextrose, sucrose, mannitol, sorbitol, cellulose and/or glycine) and lubricant (e.g., silica, talc, stearic acid and its magnesium or calcium salts and/or polyethylene glycol) in addition to the active ingredients. The tablet may include a binder such as magnesium aluminum silicate, starch paste, gelatin, tragacanth, methylcellulose, sodium carboxymethylcellulose and/or polyvinylpyrrolidine, and may optionally further contain disintegrants such as starch, agar, alginic acid or sodium salts thereof, absorbent, colorant, flavoring agent and/or sweetener. The formulation may be prepared by conventional mixing, granulating or coating methods.

In addition, the pharmaceutical composition of the present disclosure may further include an auxiliary such as preservative, wetting agent, emulsification accelerator, salt or buffer for osmotic pressure adjustment, and other therapeutically useful substance and may be formulated according to conventional methods.

The pharmaceutical composition according to the present disclosure may be administered through several routes, including orally, transdermally, subcutaneously, intravenously, or intramuscularly, and the dosage of the active ingredient may be appropriately selected depending on several factors such as the route of administration, the patient's age, gender, weight, and patient's severity. Further, the composition of the present disclosure may be administered in combination with known compounds that may enhance the desired effect.

The pharmaceutical composition according to the present disclosure may be administered to humans and animals orally or parenterally, such as intravenously, subcutaneously, intranasally, or intraperitoneally. Oral administration also includes sublingual application. Parenteral administration includes injection methods such as subcutaneous injection, intramuscular injection, and intravenous injection and drip method.

Meanwhile, the present disclosure may be used as the food composition for preventing or alleviating one or more diseases selected from the group consisting of cancer, rheumatoid arthritis, Parkinson's disease, autoimmune disease, skin disease, non-alcoholic steatohepatitis, aortic valve constriction, and cerebrovascular disease, the food composition including the adamantyl derivative, or foodologically acceptable salt thereof as an active ingredient. When the adamantyl derivative is used as an additive to a food composition, it may be added as is or used together with other foods or food ingredients and may be used appropriately according to conventional methods. In general, when preparing food or beverage, the composition of the present disclosure is added in an amount of 15% by weight or less, desirably 10% by weight or less, based on the raw material. However, for long-term intake for health and hygiene purposes or health control purposes, it may be added below the above range, and since there is no problem in terms of safety, the active ingredient may be used in an amount above the above range. That is, the mixing amount of the active ingredient may be appropriately determined depending on each purpose of use, such as prevention, health, or treatment.

The formulation of the food composition may be in the form of powder, granule, pill, tablet, capsule, as well as general food or beverage.

There are no special restrictions on the type of food. The examples of food to which the substance may be added include meat, sausage, bread, chocolate, candy, snacks, confectionery, pizza, ramen, other noodles, chewing gum, dairy products including ice cream, various soups, beverages, tea, drinks, alcoholic beverages, vitamin complexes, etc., include all health foods in a conventional sense.

The food of the present disclosure may be manufactured by methods commonly used in the industry, and during the above manufacturing process, it may be manufactured by adding raw materials and components commonly added in the industry. Specifically, it may include a protein, carbohydrate, fat, nutrient, seasoning, and flavor, and examples of the carbohydrates include glucose, fructose, maltose, sucrose, oligosaccharides, dextrin, cyclodextrin, xylitol, sorbitol, erythrol, saccharin, or synthetic flavors, but are not limited thereto.

Further, the present disclosure may be used as the cosmetic composition for preventing or alleviating one or more diseases selected from the group consisting of cancer, rheumatoid arthritis, Parkinson's disease, autoimmune disease, skin disease, non-alcoholic steatohepatitis, aortic valve constriction, and cerebrovascular disease, the food composition including the adamantyl derivative, or cosmetically acceptable salt thereof as an active ingredient.

In the present disclosure, the term “cosmetic composition” may be manufactured in the form of a general emulsified formulation or solubilized formulation. The emulsified formulations include a nourishing lotion, cream, essence, etc., and the solubilized formulations include a soft lotion, etc. The cosmetic composition may be prepared as a solution, suspension, emulsion, paste, gel, cream, lotion, powder, soap, surfactant-containing cleansing, oil, ampoule, powder foundation, emulsion foundation, wax foundation or spray, etc., but is not limited thereto. Specifically, it may be prepared as a basic cosmetic composition (lotion, cream, essence, face wash such as cleansing foam and cleansing water, pack, body oil), color cosmetic composition (foundation, lipstick, mascara, makeup base), hair product composition (shampoo, conditioner, hair conditioner, hair gel), soap, and the like.

In addition, the cosmetic composition may additionally include one or more cosmetically acceptable carriers that are mixed with general skin cosmetics, and as common ingredients, for example, oil, water, surfactant, moisturizer, lower alcohol, thickener, chelating agent, colorant, preservative, fragrance, etc., may be appropriately mixed, but are not limited thereto.

The terms used in embodiments are used for descriptive purposes only and should not be construed as limiting. Singular expressions include plural expressions unless the context clearly dictates otherwise. It should be understood that as used herein, terms such as “include” or “have” are intended to designate that there is a feature, number, step, operation, component, part, or combination thereof described in the specification, but do not preclude the existence or addition of one or more other features, numbers, steps, operations, components, parts, or combinations thereof.

Unless defined otherwise, all terms used herein, including technical or scientific terms, have the same meaning as commonly understood by one of ordinary skill in the art to which these embodiments belong. Terms such as those defined in commonly used dictionaries should be interpreted as having a meaning consistent with the meaning in the context of the related art, and unless explicitly defined in this application, it is not to be construed in an ideal or overly formal sense.

Hereinafter, Examples will be described in detail with reference to the attached drawings. However, various changes may be made to Examples, so the scope of the patent application is not limited or restricted by these Examples. It should be understood that all changes, equivalents, or substitutes for Examples are included in the scope of rights.

### Mode for Carrying Out the Invention

1. Preparation of adamantyl derivatives of the present disclosure

All chemical reagents were used as commercially available. Silica gel column chromatography was performed on silica gel 230 to 400 mesh, Merck. ¹H NMR spectra were recorded on a Bruker Avance III 400 MHz and a Bruker Fourier 300 MHz, and TMS was used as an internal standard.

### 1.1. Preparation of adamantyl derivative KUgt 102

The synthesis process of the adamantyl derivative KUgt 102 (Chemical Formula 2-4) of the present disclosure is shown in Reaction Equation 1 below.

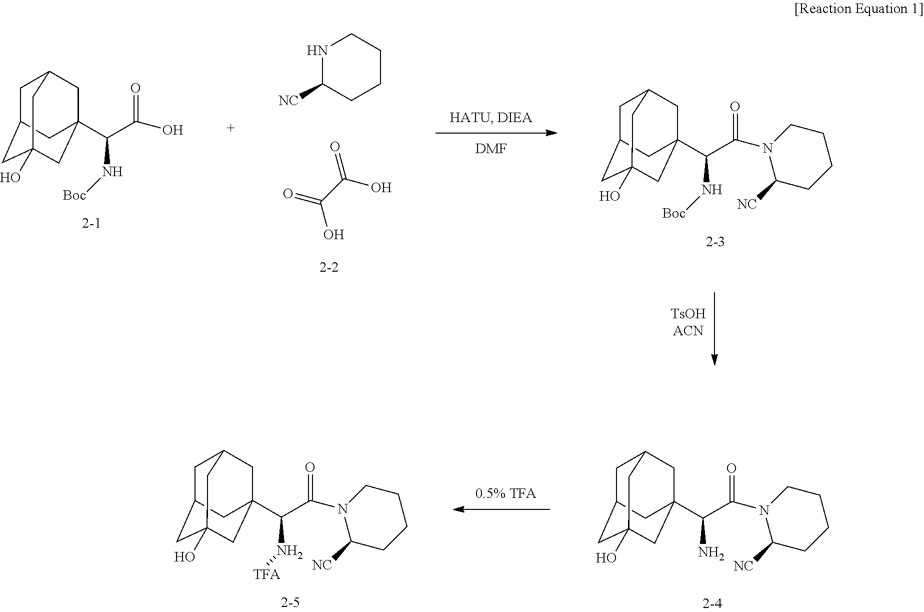

### 1.1.1. Synthesis of tert-butyl ((S)-2-((S)-2-cyanopiperidin-1-yl)-1-((1r,3R,5R,7S)-3-hydroxyadamantan-1-yl)-2-oxoethyl)carbamate (2-3)

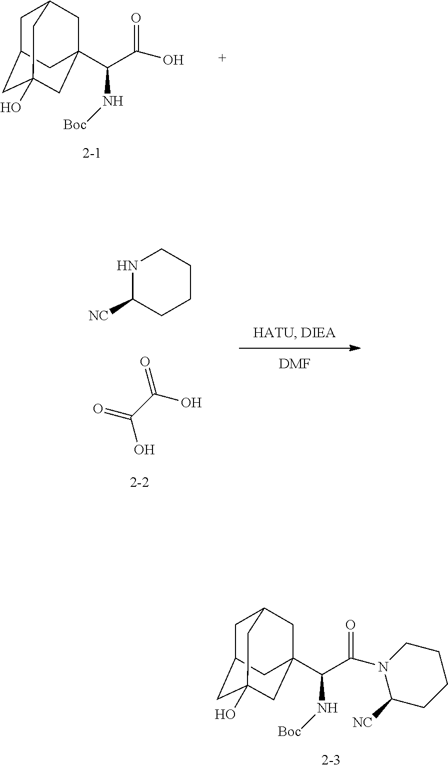

(S)-2-((tert-butoxycarbonyl)amino)-2-((1r,3R,5R,7 S)-3 -hydroxy adamantan-1-yl)acetic acid (2-1) (488 mg, 1.50 mmol, 1 eq), (S)-piperidine-2-carbonitrile oxalatet (2-2) (300 mg, 1.50 mmol, 1 eq), HATU (855 mg, 2.25 mmol, 1.5 eq) and DIEA (581 mg, 4.50 mmol, 3.0eq) were stirred in DMF (10 mL) at room temperature overnight. The mixture was concentrated, and the filtrate was purified by column chromatography on silica gel (petroleum ether/ethyl acetate=1/1) to obtain tert-butyl ((S)-2-((S)-2-cyanopiperidin-1-yl)-1-((1R,3R,5R,7S)-3-hydroxyadamantan-1-yl)-2-oxoethyl)carbamate (2-3) (190 mg, 30%), as a colorless solid.

### 1.1.2. Synthesis of (S)-1-((S)-2-amino-2-((1r,3R,5R,7S)-3-hydroxyadamantan-1-yl)acetyl)piperidine-2-carbonitrile (2-4) and trifluoroacetic acid salt (TFA) (2-5) thereof

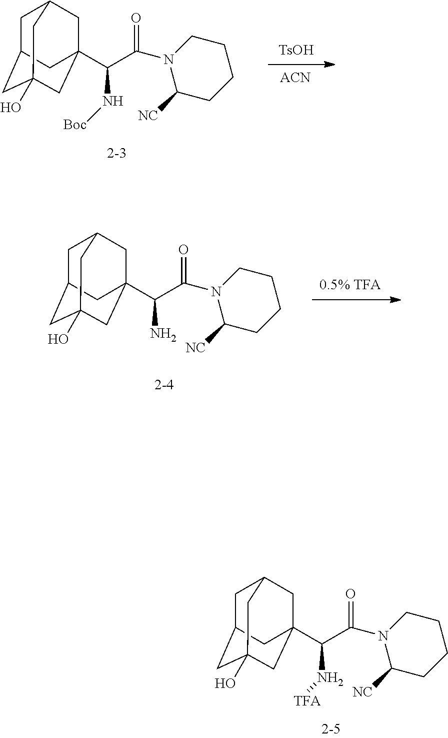

Tert-butyl ((S)-2-((S)-2-cyanopiperidin-1-yl)-1 -((1r,3R,5R,7 S)-3-hydroxyadamantan -1-yl)-2-oxoethyl)carbamate (2-3) (160 mg, 0.38 mmol, 1 eq) and TsOH (131 mg, 0.76 mmol, -2.0eq) were stirred in ACN (10 mL) at 50° C. for 6 hours. The mixture was concentrated, and the filtrate was separated and purified by column chromatography using a mixed solvent of ethyl acetate and n-hexane to obtain KUgt-102 (2-4), an oily liquid, and it was treated with a 0.5% TFA solution and then purified by prep-HPLC to obtain the compound (2-5) (66 mg, 54%) as a white solid.

¹H NMR (400 MHz, DMSO-d6): δ8.09-8.05 (m, 3H), 5.89-5.67 (m, 1H), 4.49-4.19 (m, 3H), 3.15-2.67 (m, 1H), 2.13-1.35 (m, 19H).

LCMS (Xbridge C18 5 μm, 4.6×50 mm, mobile phase: mobile phase:B(ACN), A(0.02% NH₄Ac aq); Purity>95%, Rt=2.844 min; MS Calcd.: 317; MS Found: 318 ([M+1]+).

### 1.2. Preparation of adamantyl derivative KUgt-103

The synthesis process of the adamantyl derivative KUgt 103 (Chemical Formula 3-5) of the present disclosure is shown in Reaction Equation 2 below.

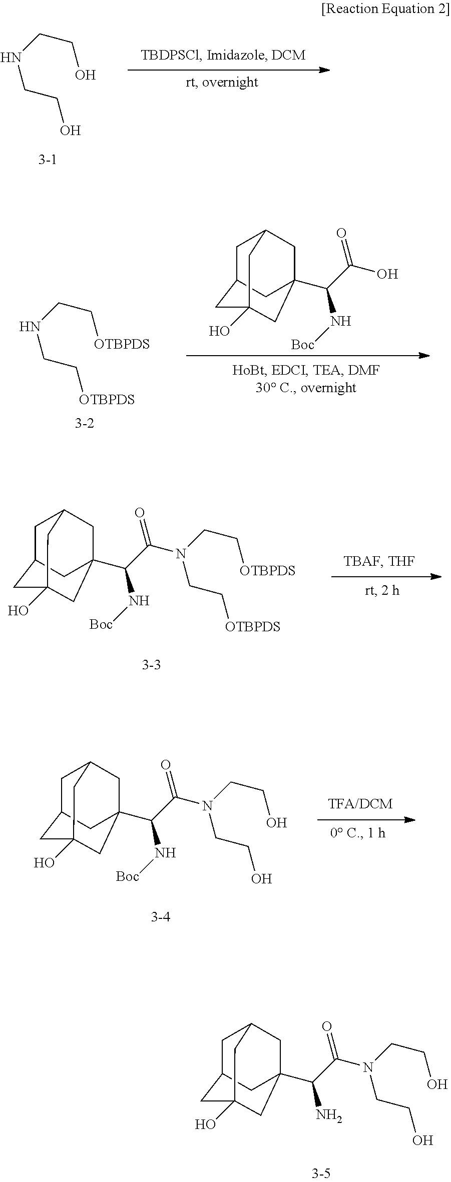

### 1.2.1. Systhesis of bis(2-((tert-butyldiphenylsilyl)oxy)ethyl)amine (3-2)

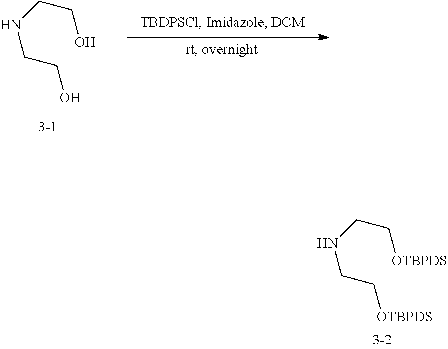

TBDPSC1 (3.45 g, 12.6 mmol, 2.2 eq) was added in a solution of 2,2′-azanediyldiethanol (3-1) (600 mg, 5.7 mmol, 1 eq) and imidazole (1.16 g, 17.1 mmol, 3 eq) in DCM (20 mL). The mixture was stirred at room temperature overnight, then poured into water and extracted with DCM. The combined organic layer was concentrated, and the filtrate was purified by column chromatography on silica gel (EA/PE=1/3, v/v) to obtain bis(2-((tert -butyldiphenylsilyl)oxy)ethypamine (3-2) (2.6 g, 79%), as a colorless oil.

### 1.2.2. Synthesis of tert-butyl ((S)-2-(bis(2-((tert -butyldiphenylsilyl)oxy)ethyl)amino)-1-((1r,3R,5R,7S)-3-hydroxyadamantan-1-yl)-2-oxoethyl)carbamate (3-3)

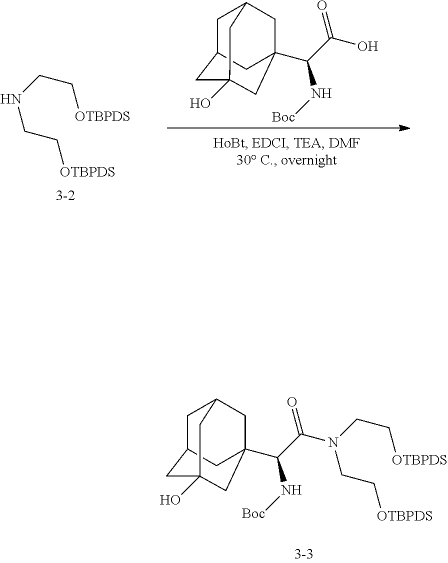

HoBt (628 mg, 4.65 mmol, 1.5 eq) and EDCI (893 mg, 4.65 mmol, 1.5 eq) were added in a solution of (S)-2-((tert-butoxycarbonyl)amino)-2-((1r,3R,5R,7S)-3-hydroxyadamantan-1-yl)acetic acid (2-1) (1.0 g, 3.1 mmol, 1 eq), bis(2-((tert-butyldiphenylsilyl)oxy)ethypamine (3-2) (2.0 g, 3.4 mmol, 1 eq), and TEA (939 mg, 9.3 mmol, 3 eq) in DMF (20 mL). The mixture was stirred at 30° C. overnight, then poured into water and extracted with EA. The combined organic layer was concentrated, and the filtrate was purified by column chromatography on silica gel (EA/PE=1/2, v/v) to obtain tert-butyl ((S)-2-(bis(2-((tert -butyldiphenylsilyl)oxy)ethyl)amino)-1-((1r,3R,5R,7S)-3-hydroxyadamantan-1-yl)-2-oxoethyl)carbamate (3-3) (2.1 g, 78%), as a white solid.

### 1.2.3. Synthesis of tert-butyl ((S)-2-(bis(2-hydroxyethyl)amino)-1-((1r,3R,5R,7S)-3-hydroxyadamantan-1-yl)-2-oxoethyl)carbamate (3-4)

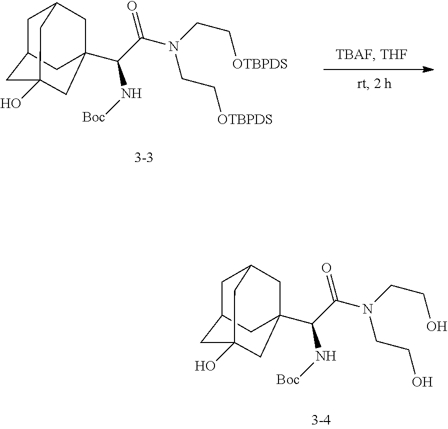

TBAF (316 mg, 1.21 mmol, 1.1 eq) was added in a solution of tert-butyl ((S)-2-(bis(2-((tert-butyl diphenylsilyl)oxy)ethyl))amino)-1-((1r,3R,5R,7S)-3-hydroxyadamantan-1 -yl)-2-oxoethyl)carbamate (3-3) (1.0 g, 1.1 mmol, 1 eq) in THF (20 mL). The mixture was stirred at room temperature for 2 hours, then poured into water and extracted with EA. The combined organic layer was concentrated, and the filtrate was purified by column chromatography on silica gel (DCM/MeOH=20/1, v/v) to obtain tert-butyl ((S)-2-(bis(2-hydroxyethyl)amino)-1-((1r,3R,5R,7S)-3 -hydroxyadamantan-1-yl)-2-oxoethyl)carbamate (3-4) (220 mg, 78%), as a white solid.

### 1.2.4. Synthesis of tert-butyl (S)-2-amino-2-((1r,3R,5R,7S)-3-hydroxyadamantan-1-yl)-N,N-bis(2-hydroxyethyl)acetamide (3-5)

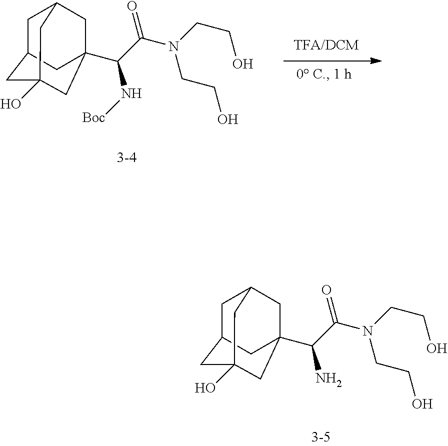 [SMILES: CC(C)(C)OC(=O)N[C@H](C(=O)N(CCO)CCO)C12CC3CC(CC(O)(C3)C1)C2 ; N[C@H](C(=O)N(CCO)CCO)C12CC3CC(CC(O)(C3)C1)C2]

TFA (2 mL) was added in a solution of tert-butyl ((S)-2-(bis(2-hydroxyethyl)amino) -((1r,3R,5R,7S)-3-hydroxyadamantan-1-yl)-2-oxoethyl)carbamate (3-4) (220 mg, 0.53 mmol, 1 eq) in DCM (2 mL) at 0° C. The mixture was stirred at 0° C. for 1 hour and then concentrated. The filtrate was purified by prep-HPLC to obtain (S)-2-amino-2-((1r,3R,5R,7S)-3-hydroxyadamantan-1-yl)-N,N-bis(2-hydroxyethyl)acetamide (3-5) (70 mg, 42%), as a white solid.

¹H NMR (400 MHz, DMSO-d6): δ7.95 (s, 3H), 4.51-4.78 (m, 3H), 4.07 (s, 1H), 3.73-3.85 (m, 2H), 3.46-3.59 (m, 4H), 3.25-3.31 (m, 1H), 3.05-3.12 (m, 1H), 2.12 (s, 2H), 1.41-1.58 (m, 12H).

MS Calcd.: 312.2, MS Found: 313.2 ([M+H]+).

### 25 2. Measurement of DPP4 inhibitory activity according to treatment with adamantyl derivative of the present disclosure

DPP4 screening assay was performed to identify the DPP4 inhibitory activity of the adamantyl derivatives KUgt-102 and KUgt-103 of the present disclosure. Abcam (Boston, USA) 133081 was used as an assay kit, and Gly-Pro-Aminomethylcoumarin was used as a fluorescent substrate. That is, the fluorescence emitted from aminomethylcoumarin (AMC), which was freed by cleaving the peptide bond by DPP, was analyzed at an excitation wavelength of 350 to 360 nm and an emission wavelength of 450 to 465 nm to measure the DPP4 inhibitory activity.

More specifically, DPP Assay Buffer (10×) was dissolved in HPLC-grade pure water, thereby preparing 1× solution. 120 μl of DPP4 (human recombinant) contained in each glass vial was mixed with 480 μl of 1× assay buffer. 120 μl of Gly-Pro-AMC, in which 5 mM H-Gly-Pro was conjugated to AMC, was dissolved in 2.88 ml assay buffer to produce DPP substrate. As a positive control, vildagliptin and saxagliptin, which have a similar structure to the derivative of the present disclosure, known as DPP4 inhibitor, were dissolved in 500 μl of assay buffer to make 1 mM, and the result was used in the reaction. As an experimental group, 10 μl each of adamantyl derivatives KUgt-102 and KUgt-103 was used in the reaction. The control or experimental group was added as shown in Table 1 below, and then the mixture reacted at 37° C. for 30 minutes. The reaction was repeated three times.

TABLE 1

| | Assay | | | | |
|---|---|---|---|---|---|
| | Buffer | DPP | Solvent | Sample | Substrate |
| 100% Initial activity | 30 μl | 10 μl | 10 μl | — | 50 μl |
| Background | 40 μl | — | 10 μl | — | 50 μl |
| Sitagliptin | 30 μl | 10 μl | — | 10 μl | 50 μl |
| Sample | 30 μl | 10 μl | — | 10 μl | 50 μl |

After the reaction, fluorescence was measured at an excitation wavelength of 350-360 nm and an emission wavelength of 450-465 nm using a fluorescence microplate reader (SPECTRA MAX Gemini EM, USA). Thereafter, the inhibitory activity was calculated as follows.

$$\% Inhibition = [ \frac{Initial Activity - Inhibitor}{Initial Activity} ] \times 100$$

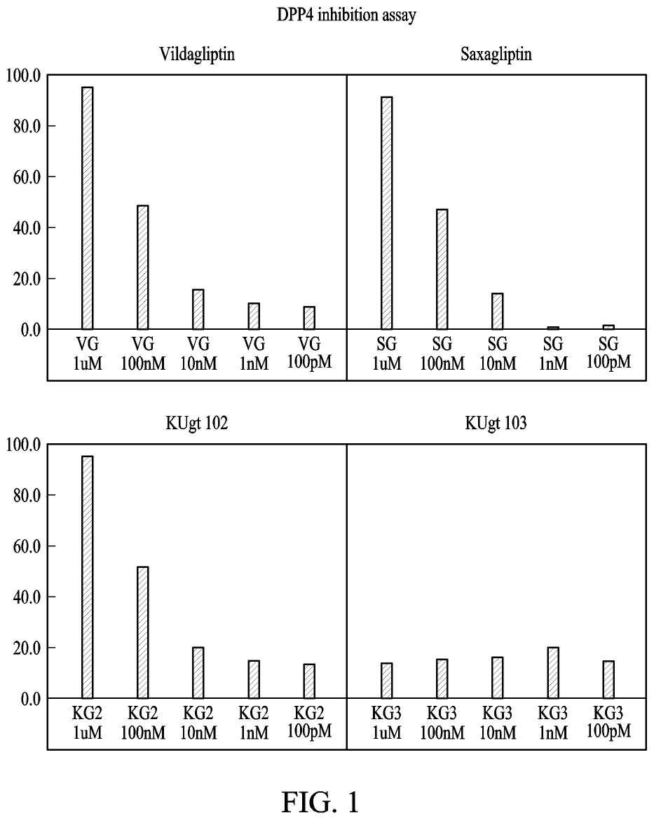

As a result of the analysis of the inhibitory activity, the adamantyl derivatives of the present disclosure, KUgt-102 and KUgt-103, all showed excellent DPP4 inhibitory activity, in particular, KUgt-102, as a positive control, showed higher inhibitory activity than vildagliptin and saxacliptin, DPP4 inhibitors with similar structures (See FIG. 1).

### 3. Measurement of lung cancer cell line viability according to treatment with adamantyl derivative of the present disclosure

After treating LLC, a mouse lung carcinoma cell line, and A549, a human lung cancer cell line, with KUgt-102 derivatives, cell viability was measured by water soluble tetrazolium (WST) cell viability assay.

The lung cancer cell lines LLC and A549 were attached to a 96-well plate at 5×10e³ and 1×10e⁴ cells/wells, respectively. The next day, the culture medium was removed, and they were added with new medium treated with the KUgt-102 derivative and evogliptin and saxagliptin, respectively, which have a structure similar to that of the derivative of the present disclosure known as a DPP4 inhibitor, as a positive control, in a concentration-dependent manner from 0.01 to 20 mM. Cells treated with the derivative and control group were cultured at 37° C. in 5% CO₂. After 2 days, the cultured cells were treated with WST reagent (EZ-CYTOX, Dogen, Republic of Korea) at a final volume of 1:10 to analyze cell viability. Thereafter, the cultured plate was stored in an incubator at 37° C.

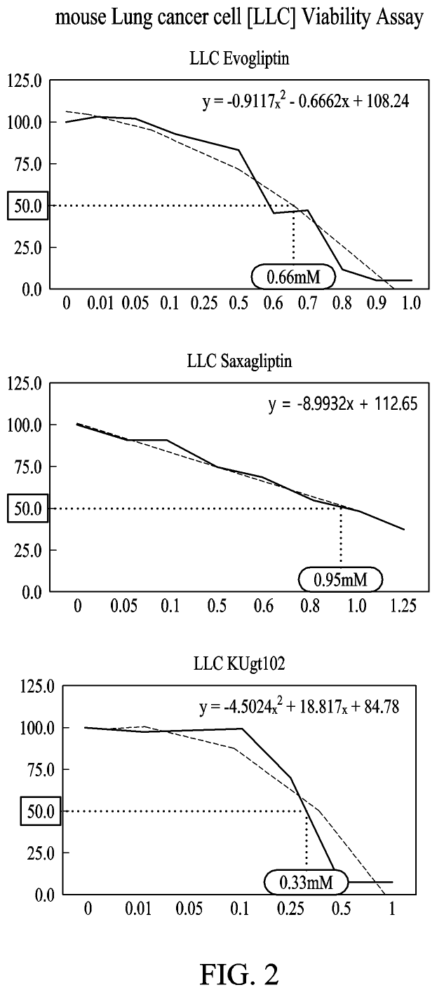

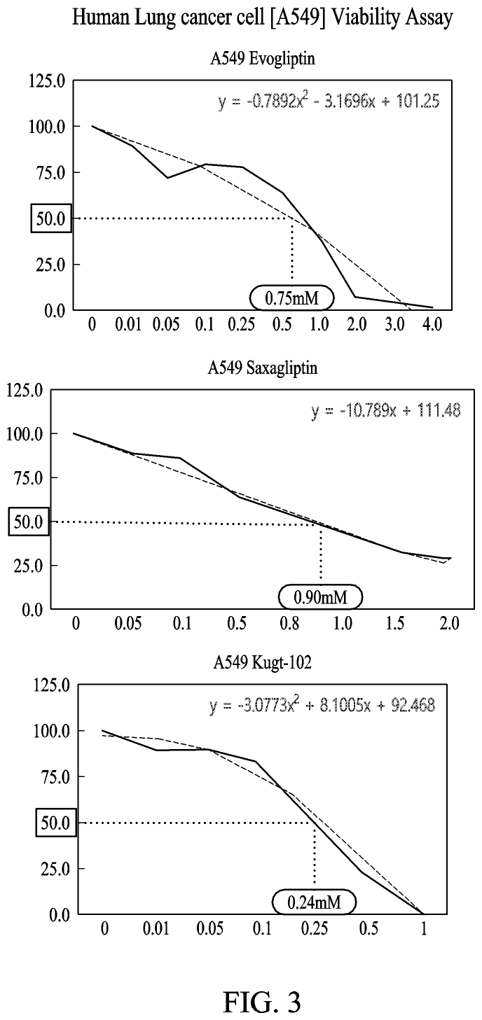

After culturing for 1 hour, the color change was measured at 450 nm using a microplate reader (SpectraMAX190, molecular Devices, CA, USA), and the measurement results are shown in FIGS. 2 and 3.

As a result, it was confirmed that the adamantyl derivative KUgt-102 inhibited cell viability in a concentration-dependent manner in the lung cancer cell lines LLC and A549. In particular, in human lung cancer cell lines, the KUgt-102 derivative showed an effective killing effect on lung cancer cell lines in an amount of about 30% or less compared to evogliptin and saxagliptin, which have a similar structure to the derivative of the present disclosure, known as DPP4 inhibitor. (See Table 2) That is, based on the same amount, the KUgt-102 derivative of the present disclosure showed about three times better anticancer activity.

TABLE 2
Cell growth inhibition IC50 (mM)

| | Cell | | | |
|---|---|---|---|---|
| Cell type | line | Evogliptin | Saxagliptin | KUgt-102 |
| Mouse Lung | LLC | 0.66 ± 0.01 | 0.95 ± 0.01 | 0.33 ± 0.01 |
| carcinoma | | | | |
| Human Lung | A549 | 0.75 ± 0.01 | 0.90 ± 0.01 | 0.24 ± 0.01 |
| cancer | | | | |

Although the embodiments have been described with limited drawings as described above, one or ordinary skill in the art can apply various technical modifications and variations based on the above. For example, adequate results may be achieved even though the techniques described may be performed in a different order than the method described, and/or components of the described system, structure, device, circuit, etc. are coupled or combined in a form different from the method described or replaced or substituted by another component or equivalent.

Therefore, other implementations, other embodiments, and equivalents to the claims also fall within the scope of the claims described below.

### INDUSTRIAL APPLICABILITY

The present disclosure relates to a novel adamantyl derivative or a pharmaceutically acceptable salt thereof and its use, the adamantyl derivative may be a DPP4 (dipeptidyl peptidase-4) inhibitor, and may be useful in the field of prevention, alleviation, or treatment of cancer, rheumatoid arthritis, Parkinson's disease, autoimmune disease, skin disease, non-alcoholic steatohepatitis, aortic valve constriction, or cerebrovascular disease. In particular, the adamantyl derivative inhibits DPP4 in the tumor microenvironment (TME) to attract T cells to tumor tissue, thereby killing cancer cells, which may be used in the field of prevention, alleviation, or treatment of cancer. The cancer may be selected from the group consisting of prostate cancer, thyroid cancer, kidney cancer, carcinoma, endometrial cancer, lung cancer, urinary epithelial cancer, colorectal cancer, breast cancer, rectal cancer, cervical cancer, glioma, colon cancer, head and neck cancer, stomach cancer, liver cancer, pancreatic cancer, testicular cancer, ovarian cancer, blood cancer, skin cancer, brain tumor, and the like.

1. An adamantyl derivative represented by the following [Chemical Formula 1], a racemate, isomer, solvate or pharmaceutically acceptable salt thereof: 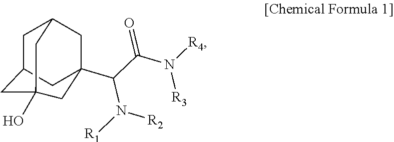
in the above [Chemical Formula 1],
R₁ and R₂ are the same or different from each other and are each independently hydrogen, or a C₁-C₈ chain or a cyclic alkyl group;
R₃ and R₄ are the same or different from each other, and are each independently a C₁-C₈ chain or cyclic alkyl group, a C₁-C₈ alkoxy group, or a hydroxy group, or
combine with each other to form a C₃-C₈ single ring heterocycloalkyl group containing N;
one or more hydrogens of the C₁-C₈ chain or cyclic alkyl group, C₁-C₈ alkoxy group, or C₃-C₈ single ring heterocycloalkyl group are each independently unsubstituted or substituted with any one or more substituents selected from the group consisting of a halogen group, a hydroxy group, a cyano group, a nitro group, and a trifluoromethyl group.

2. The adamantyl derivative, racemate, isomer, solvate or pharmaceutically acceptable salt thereof of claim 1, wherein R₂ and R₃ are independently a C₁-C₈ chain alkyl group substituted with a hydroxy group.

3. The adamantyl derivative, racemate, isomer, solvate or pharmaceutically acceptable salt thereof of claim 1, wherein the single ring heterocycloalkyl group formed by combining R₂ and R₃ is substituted with a cyano group or a trifluoromethyl group.

4. The adamantyl derivative, racemate, isomer, solvate or pharmaceutically acceptable salt thereof of claim 1, wherein the adamantyl derivative represented by [Chemical Formula 1] is any one selected from the group consisting of compounds represented by the following formulas:

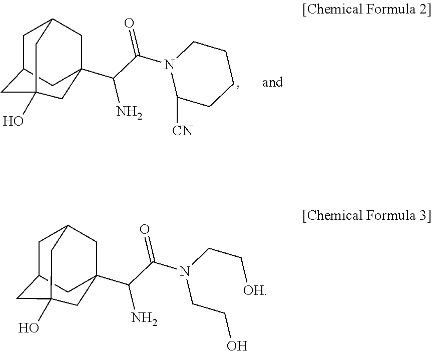 [SMILES: N#CC1CCCCN1C(=O)C(N)C12CC3CC(CC(O)(C3)C1)C2 ; NC(C(=O)N(CCO)CCO)C12CC3CC(CC(O)(C3)C1)C2]

5. The adamantyl derivative, racemate, isomer, solvate or pharmaceutically acceptable salt thereof of claim 1, wherein the adamantyl derivative or the pharmaceutically acceptable salt thereof inhibits dipeptidyl peptidase-4 (DPP4).

6. (canceled)

7. A pharmaceutical composition for preventing or treating one or more diseases selected from the group consisting of cancer, rheumatoid arthritis, Parkinson's disease, autoimmune disease, skin disease, non-alcoholic steatohepatitis, aortic valve constriction, and cerebrovascular disease, the pharmaceutical composition comprising the adamantyl derivative, racemate, isomer, solvate or pharmaceutically acceptable salt thereof of claim 1 as an active ingredient.

8. The pharmaceutical composition of claim 7, wherein the cancer comprises any one selected from the group consisting of prostate cancer, thyroid cancer, kidney cancer, carcinoma, endometrial cancer, lung cancer, urinary epithelial cancer, colorectal cancer, breast cancer, rectal cancer, cervical cancer, glioma, colon cancer, head and neck cancer, stomach cancer, liver cancer, pancreatic cancer, testicular cancer, ovarian cancer, blood cancer, skin cancer, or brain tumor.

9. A method for preventing or treating one or more diseases selected from the group consisting of cancer, rheumatoid arthritis, Parkinson's disease, autoimmune disease, skin disease, non-alcoholic steatohepatitis, aortic valve constriction, and cerebrovascular disease, the method comprising administering the adamantyl derivative, racemate, isomer, solvate or pharmaceutically acceptable salt thereof of claim 1 to a subject.

10. The method of claim 9, wherein the cancer comprises any one selected from the group consisting of prostate cancer, thyroid cancer, kidney cancer, carcinoma, endometrial cancer, lung cancer, urinary epithelial cancer, colorectal cancer, breast cancer, rectal cancer, cervical cancer, glioma, colon cancer, head and neck cancer, stomach cancer, liver cancer, pancreatic cancer, testicular cancer, ovarian cancer, blood cancer, skin cancer, or brain tumor.

11.-14. (canceled)
